(19) United States
(12) Patent Application Publication
(10) Pub. No.: US 2023/0263468 A1
(43) Pub. Date: Aug. 24, 2023

(54) An Electronics Module for a Wearable Article, a Controller for an Electronics Module, and a Wearable Article Incorporating an Electronics Module

(71) Applicant: Prevayl Innovations Limited, Manchester, Greater Manchester (GB)

(72) Inventor: Michael John Lynch, Cheshire (GB)

(21) Appl. No.: 18/006,441

(22) PCT Filed: Jul. 29, 2021
(86) PCT No.: PCT/GB2021/051953

§ 371 Date: Jan. 23, 2023

(30) Foreign Application Priority Data
Jul. 30, 2020 (GB) 2011889.9

(51) Int. Cl. A61B 5/00; G16H 40/63

(52) U.S. Cl. CPC A61B 5/6804; G16H 40/63

(57) ABSTRACT

An electronics module (100) for a wearable article (200) comprises a controller. The electronics module receives biosignals such as ECG signals from sensors on the wearable article and processes these signals to provide data and information to a user. The controller is operable to apply filters to the incoming biosignals that have been digitised and processed by an analogue to digital converter. The filters are provided to remove, or at least mitigate, noise from the digital signal. An example of noise that could be advantageously removed is electromagnetic interference at mains frequencies. Mains frequencies are typically 50 Hz or 60 Hz depending upon location. The controller is configured to apply a suitable filter depending upon the location, which is determined from a GPS signal from a GPS device on the electronics module or on a remote mobile device (100). This has the advantage of being able to easily and quickly select and apply the relevant filter depending upon the location.

The present invention is directed towards an electronics module for a wearable article. More particularly, the wearable article comprises a biosignal measuring apparatus for sensing biosignals from a wearer of the wearable article, and which incorporates a sensor assembly and the electronics module. The electronics module is arranged to transmit biosignal data to a mobile or remote device. The present invention is also directed towards a controller for an electronics module and a wearable article incorporating an electronics module.

### BACKQROUND

Wearable articles, such as garments, incorporating sensors are wearable electronics used to measure and collect information from a wearer. Such wearable articles are commonly referred to as ‘smart clothing’. It is advantageous to measure biosignals of the wearer during exercise, or other scenarios.

It is known to provide a garment, or other wearable article, to which an electronic device (i.e. an electronic module, and/or related components) is attached in a prominent position, such as on the chest or between the shoulder blades. Advantageously, the electronic device is a detachable device. The electronic device is configured to process the incoming signals, and the output from the processing is stored and/or displayed to a user in a suitable way

A sensor senses a biosignal such as electrocardiogram (ECG) signals and the biosignals are coupled to the electronic device, via an interface.

The sensors may be coupled to the interface by means of conductors which are connected to terminals provided on the interface to enable coupling of the signals from the sensor to the interface.

Electronics modules for wearable articles such as garments are known to communicate with mobile devices over wireless communication protocols such as Bluetooth® and Bluetooth® Low Energy. These electronics modules are typically removably attached to the wearable article, interface with internal electronics of the wearable article, and comprise a Bluetooth® antenna for communicating with the mobile device.

The electronic device includes drive and sensing electronics comprising components and associated circuitry, to provide the required functionality.

The drive and sensing electronics include a power source to power the electronic device and the associated components of the drive and sensing circuitry.

ECG sensing is used to provide a plethora of information about a person's heart. It is one of the simplest and oldest techniques used to perform cardiac investigations. In its most basic form, it provides an insight into the electrical activity generated within heart muscles that changes over time. By detecting and amplifying these differential biopotential signals, a lot of information can be gathered quickly, including the heart rate. Among professional medical staff, individual signals have names such as “the QRS complex,” which is the largest part of an ECG signal and is a collection of Q, R, and S signals, including the P and T waves.

Sensors that are used to measure, for example, ECG signals and make contact with skin may be of wet or dry type. Typically, for clinical use, they are wet electrodes that use a composition of a watery polymerized hydrogel and adhere to the body. In non-clinical applications, the electrodes are usually dry. Electrodes are typically two sensors integrated or fixed to into an elasticated and conductive material that connects to a set of drive electronics. Any electrode requires a good connection to the body to provide a reliable signal of sufficient amplitude for detection.

Electrode size and material characteristics also influence the signal quality and levels detected. While the use of dry electrodes is far more convenient than using wet types (you can just take them on and off), dry electrodes present a very high impedance when initially placed on the body. This means that the ECG signal is likely to be attenuated, resulting in a small signal. This ‘dry start’ scenario typically lasts for a short duration until the wearer has exercised sufficiently to start sweating, thus lowering the impedance and increasing signal levels. To accommodate dry starts, the input impedance of the analog circuitry of the ECG channel through the ECG signal is coupled should be very high so that attenuation is kept to a minimum.

If sending ECG signals during exercise, as the body moves during exercise there are several factors that can interfere with signal quality. For example, the simple motion of a body running or cycling, the movement of clothes hitting the body and/or the chest strap, and the movement of the electrodes all result in interference to the ECG signal. Removing such interference from these motion artefacts is essential if the ECG signal quality is to be maintained.

Typically, such motion artefacts will be present on, or common to, signals from both electrode pads, so the common mode rejection ratio (CMRR) of the analogue to digital converter which converts the analogue ECG signal to a digital signal for further processing needs to be as high as possible. Also, it should be observed that the heavier the sensor electronics, the more likely it is that the unit will bounce around when in use, creating more motion artefacts.

Another source of noise in an ECG signal is that caused by electromagnetic interference (EMI) in the environment resulting particularly from alternating current flowing in nearby cables and wiring, for example in a mains or utility power supply. Typically, the mains power supply oscillates at a utility frequency (also called utility frequency of lines frequency) which is the nominal frequency of the oscillations of alternating current (AC). The utility frequency may be at 50 Hz or 60 Hz depending upon location. EMI at these frequencies can be particularly problematic when detecting ECG signals.

To address this, filters are usually employed to remove unwanted noise at these frequencies. Notch filters are often used for this purpose with different filters for the different frequencies. Users may be able to select the relevant notch filter for the environment. Some systems are able to detect the frequency and apply the appropriate filter. An alternative is to apply a single filter covering both 50 Hz and 60 Hz.

This can be problematic as the ability to detect which filter to apply can waste valuable processing time and power. There may be times where the signal is incorrectly filtered.

An object of the present invention is to provide an improved electronic device for a wearable article with noise filtering, particularly for filtering EMI noise.

### SUMMARY

According to a first aspect of the present invention, there is provided a method. The method is performed by a controller for an electronics module for a wearable article the electronics module further including an interface, coupled to the controller, and arranged to receive signals from a sensor unit. The method further comprises determining the location of the electronics module. The method further comprises selecting a filter for the controller to apply to the received signal based on the determined location.

This provides an advantage of being able to apply the correct filter to the incoming biosignals to filter out unwanted artefacts such as EMI or motion artefacts thus minimising the risk of applying unwanted or incorrect filters whilst reducing time at start up and the beginning of any monitoring period.

The method may comprise determining a likely frequency of electromagnetic interference present in a received signal from the determined location. The method may further comprises selecting the filter for the controller based on the determined location and the determined likely frequency.

The method may comprise determining a likely use of the electronic module from the determined location. The method may comprise selecting the filter for the controller based on the determined location and the determined likely frequency.

The likely use may be determined to be in an outside setting. The method may further comprise deselecting a filter in response to determining a location of an outside setting.

The method may comprise accessing a look up table stored in memory on the electronics module to determine the likely frequency for the determined location.

The likely frequency may be a utility frequency.

The location may be derived from a location device. The location device may be provided on the electronics module. Alternatively, the location is derived from a location device provided on a mobile device in communication with the electronics module. The method may include the step of receiving the predetermined location data from the mobile device prior to determining the likely frequency of electromagnetic interference present in a received signal from the determined location.

The location data may be derived from data obtained from the Global Positioning System.

In accordance with a second aspect of the present invention, there is provided an electronics module for a wearable article. The electronics module comprises a controller and an interface, coupled to the controller, and arranged to receive signals from a sensor unit. The controller is further configured to determine the location of the electronics module. The controller is further configured to select a filter for the controller to apply to the received signal based on the determined location.

The controller may be further configured to determine a likely frequency of electromagnetic interference present in a received signal from the determined location. The controller may be further configured to select the filter for the controller based on the determined location and the determined likely frequency.

The controller may be further configured to determine a likely use of the electronic module from the determined location. The controller may be further configured to select the filter for the controller based on the determined location and the determined likely frequency.

The likely use may be determined to be in an outside setting. The controller may be further configured to deselect a filter in response to determining a location of an outside setting.

The filter may be a notch filter. Alternatively, the filter may be a bandpass filter, a low pass filter, a high pass filter.

The electronics module may further comprise a memory. The memory may include a look up table. The controller may be configured to access the memory to determine the likely frequency for the determined location.

The likely frequency may be a utility frequency.

The electronics module may further comprise a location device coupled to the controller and arranged to provide location data to the controller. The controller may be configured to determine the location from the location data.

The location device may be a Global Navigation Satellite System (GNSS) device.

The electronics module for a wearable article may further comprise a communicator coupled to the controller and arranged for communication with a mobile device. The determined location may be derived from a location device provided on the mobile device.

In accordance with a third aspect of the present invention, there is provided a wearable article including an electronics module according to the second aspect of the present invention.

In accordance with a third aspect of the present invention, there is provided a controller for an electronics module for a wearable article according to the second aspect of the present invention.

### BRIEF DESCRIPTION OF THE DRAWINGS

Examples of the present disclosure will now be described with reference to the accompanying drawings, in which:

FIG. 1 shows a schematic diagram for an example system according to aspects of the present disclosure;

FIG. 2 shows a schematic diagram for an example electronics module according to aspects of the present disclosure;

FIG. 3 shows a detailed schematic diagram of the electronics components of an example electronics module according to aspects of the present disclosure;

FIG. 4 shows a schematic diagram for an example analogue to digital converter used in the example electronics module of FIGS. 4 and 5 according to aspects of the present disclosure;

FIG. 5 shows a flow diagram for an example method according to aspects of the present disclosure; and

FIG. 6 shows a flow diagram for a second example method according to aspects of the present disclosure.

### DETAILED DESCRIPTION

The following description with reference to the accompanying drawings is provided to assist in a comprehensive understanding of various embodiments of the disclosure as defined by the claims and their equivalents. It includes various specific details to assist in that understanding but these are to be regarded as merely exemplary. Accordingly, those of ordinary skill in the art will recognize that various changes and modifications of the various embodiments described herein can be made without departing from the scope and spirit of the disclosure. In addition, descriptions of well-known functions and constructions may be omitted for clarity and conciseness.

The terms and words used in the following description and claims are not limited to the bibliographical meanings but are merely used by the inventor to enable a clear and consistent understanding of the disclosure. Accordingly, it should be apparent to those skilled in the art that the following description of various embodiments of the disclosure is provided for illustration purpose only and not for the purpose of limiting the disclosure as defined by the appended claims and their equivalents.

It is to be understood that the singular forms “a,” “an,” and “the” include plural referents unless the context clearly dictates otherwise.

“Wearable article” as referred to throughout the present disclosure may refer to any form of device interface which may be worn by a user such as a smart watch, necklace, garment, bracelet, or glasses. The wearable article may be a textile article. The wearable article may be a garment. The garment may refer to an item of clothing or apparel. The garment may be a top. The top may be a shirt, t-shirt, blouse, sweater, jacket/coat, or vest. The garment may be a dress, garment brassiere, shorts, pants, arm or leg sleeve, vest, jacket/coat, glove, armband, underwear, headband, hat/cap, collar, wristband, stocking, sock, or shoe, athletic clothing, personal protective equipment, swimwear, wetsuit or dry suit.

The term “wearer” includes a user who is wearing, or otherwise holding, the wearable article.

The type of wearable garment may dictate the type of biosignals to be detected. For example, a hat or cap may be used to detect electroencephalogram or magnetoencephalogram signals.

The wearable article/garment may be constructed from a woven or a non-woven material. The wearable article/garment may be constructed from natural fibres, synthetic fibres, or a natural fibre blended with one or more other materials which can be natural or synthetic. The yarn may be cotton. The cotton may be blended with polyester and/or viscose and/or polyamide according to the application. Silk may also be used as the natural fibre. Cellulose, wool, hemp and jute are also natural fibres that may be used in the wearable article/garment. Polyester, polycotton, nylon and viscose are synthetic fibres that may be used in the wearable article/garment.

The garment may be a tight-fitting garment. Beneficially, a tight-fitting garment helps ensure that the sensor devices of the garment are held in contact with or in the proximity of a skin surface of the wearer. The garment may be a compression garment. The garment may be an athletic garment such as an elastomeric athletic garment.

The garment has sensing units provided on an inside surface which are held in close proximity to a skin surface of a wearer wearing the garment. This enables the sensing units to measure biosignals for the wearer wearing the garment.

The sensing units may be arranged to measure one or more biosignals of a wearer wearing the garment.

“Biosignal” as referred to throughout the present disclosure may refer to signals from living beings that can be continually measured or monitored. Biosignals may be electrical or non-electrical signals. Signal variations can be time variant or spatially variant.

Sensing components may be used for measuring one or a combination of bioelectrical, bioimpedance, biochemical, biomechanical, bioacoustics, biooptical or biothermal signals of the wearer 600. The bioelectrical measurements include electrocardiograms (ECG), electrogastrograms (EGG), electroencephalograms (EEG), and electromyography (EMG). The bioimpedance measurements include plethysmography (e.g., for respiration), body composition (e.g., hydration, fat, etc.), and electroimpedance tomography (EIT). The biomagnetic measurements include magnetoneurograms (MNG), magnetoencephalography (MEG), magnetogastrogram (MGG), magnetocardiogram (MCG). The biochemical measurements include glucose/lactose measurements which may be performed using chemical analysis of the wearer 600's sweat. The biomechanical measurements include blood pressure. The bioacoustics measurements include phonocardiograms (PCG). The biooptical measurements include orthopantomogram (OPG). The biothermal measurements include skin temperature and core body temperature measurements.

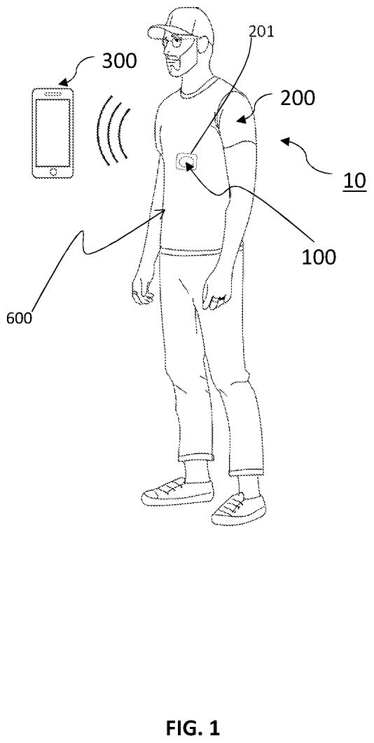

Referring to FIGS. 1 to 3, there is shown an example system 10 according to aspects of the present disclosure. The system 10 comprises an electronics module 100, a wearable article in the form of a garment 200, and a mobile device 300. The garment 200 is worn by a user who in this embodiment is the wearer 600 of the garment 200.

The electronics module 100 is arranged to integrate with sensing units 400 incorporated into the garment 200 to obtain signals from the sensing units 400. The sensing units 400 comprise one or more sensors 209, 211 with associated conductors 203, 207 and other components and circuitry. The electronics module 100 is further arranged to wirelessly communicate data to the mobile device 300. Various protocols enable wireless communication between the electronics module 100 and the mobile device 300. Example communication protocols include Bluetooth®, Bluetooth® Low Energy, and near-field communication (NFC).

The garment 200 has an electronics module holder in the form of a pocket 201. The pocket 201 is sized to receive the electronics module 100. When disposed in the pocket 201, the electronics module 100 is arranged to receive sensor data from the sensing units 400. The electronics module 100 is therefore removable from the garment 200.

The present disclosure is not limited to electronics module holders in the form pockets.

Alternatively, the electronics module 100 may be configured to be releasably mechanically coupled to the garment 200. The mechanical coupling of the electronic module 100 to the garment 200 may be provided by a mechanical interface such as a clip, a plug and socket arrangement, etc. The mechanical coupling or mechanical interface may be configured to maintain the electronic module 100 in a particular orientation with respect to the garment 200 when the electronic module 100 is coupled to the garment 200. This may be beneficial in ensuring that the electronic module 100 is securely held in place with respect to the garment 200 and/or that any electronic coupling of the electronic module 100 and the garment 200 (or a component of the garment 200) can be optimized. The mechanical coupling may be maintained using friction or using a positively engaging mechanism, for example.

Beneficially, the removable electronic module 100 may contain all the components required for data transmission and processing such that the garment 200 only comprises the sensing units 400 e.g. the sensors 209, 211 and communication pathways 203, 207. In this way, manufacture of the garment 200 may be simplified. In addition, it may be easier to clean a garment 200 which has fewer electronic components attached thereto or incorporated therein. Furthermore, the removable electronic module 100 may be easier to maintain and/or troubleshoot than embedded electronics. The electronic module 100 may comprise flexible electronics such as a flexible printed circuit (FPC).

The electronic module 100 may be configured to be electrically coupled to the garment 200.

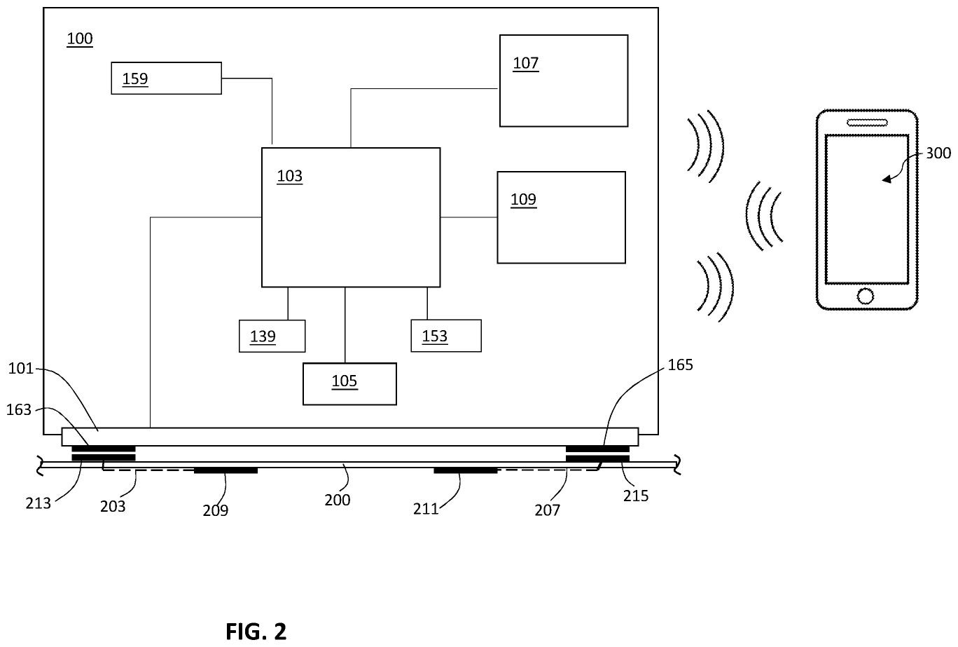

Referring to FIG. 2, there is shown a schematic diagram of an example of the electronics module 100 of FIG. 1.

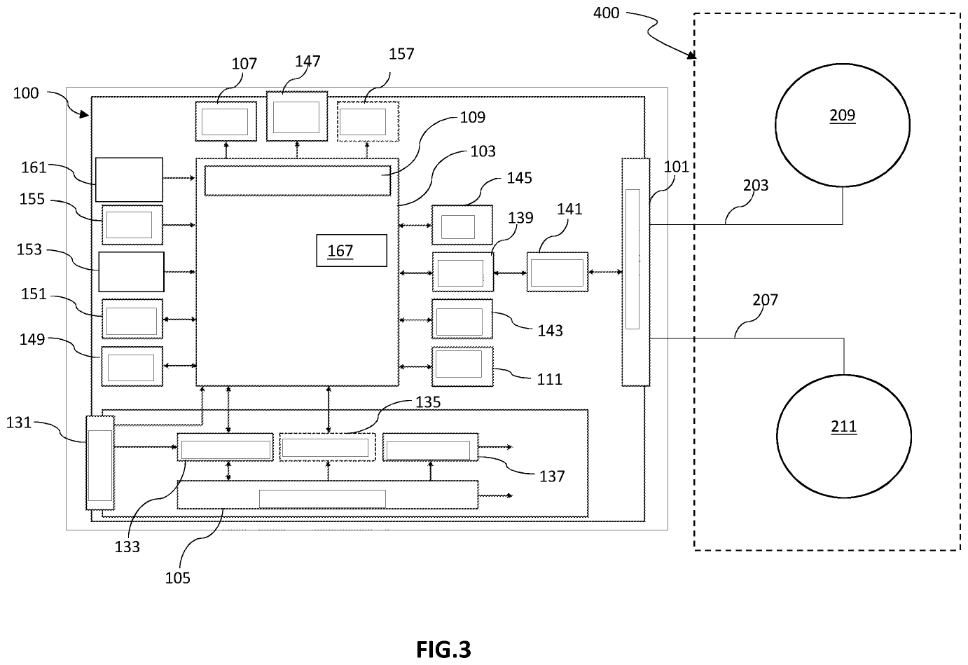

A more detailed block diagram of the electronics components of electronics module 100 and garment are shown in FIG. 3.

The electronics module 100 comprises an interface 101, a controller 103, a power source 105, and a number of communication devices which, in the exemplar embodiment comprises a first antenna 107, a second antenna 109 and a wireless communicator 159. The electronics module 100 also includes an input unit such as a proximity sensor or a motion sensor 111, for example in the form of an inertial measurement unit (IMU).

The electronics module 100 also includes several additional peripheral devices that are used to perform specific functions as will be described in further detail herein.

The interface 101 is arranged to communicatively couple with the sensing unit 400 of the garment 200. The sensing unit 400 comprises—in this example—the two sensors 209, 211 coupled to respective first and second electrically conductive pathways 203, 207, each with respective termination points 213, 215. The interface 101 receives signals from the sensors 209, 211. The controller 103 is communicatively coupled to the interface 101 and is arranged to receive the signals from the interface 101 for further processing.

The interface 101 of the embodiment described herein comprises first and second contacts 163, 165 which are arranged to be communicatively coupled to the termination points 213, 215 the respective first and second electrically conductive pathways 203, 207. The coupling between the termination points 213, 215 and the respective first and second contacts 163, 165 may be conductive or a wireless (e.g., inductive) communication coupling.

In this example the sensors 209, 211 are used to measure electropotential signals such as electrocardiogram (ECG) signals, although the sensors 209, 211 could be configured to measure other biosignal types as also discussed above.

In this embodiment, the sensors 209, 211 are configured for so-called dry connection to the wearer's skin to measure ECG signals.

The power source 105 may comprise a plurality of power sources. The power source 105 may be a battery. The battery may be a rechargeable battery. The battery may be a rechargeable battery adapted to be charged wirelessly such as by inductive charging. The power source 105 may comprise an energy harvesting device. The energy harvesting device may be configured to generate electric power signals in response to kinetic events such as kinetic events 10 performed by the wearer 600 of the garment 200. The kinetic event could include walking, running, exercising or respiration of the wearer 600. The energy harvesting material may comprise a piezoelectric material which generates electricity in response to mechanical deformation of the converter. The energy harvesting device may harvest energy from body heat of the wearer 600 of the garment. The energy harvesting device may be a thermoelectric energy harvesting device. The power source 105 may be a super capacitor, or an energy cell.

The first antenna 107 is arranged to communicatively couple with the mobile device 300 using a first communication protocol. In the example described herein, the first antenna 107 is a passive tag such as a passive Radio Frequency Identification (RFID) tag or Near Field Communication (NFC) tag. These tags comprise a communication module as well as a memory which stores the information, and a radio chip. The mobile device 300 is powered to induce a magnetic field in an antenna of the mobile device 300. When the mobile device 300 is placed in the magnetic field of the communication module antenna 107, the mobile device 300 induces current in the communication module antenna 107. This induced current is used to retrieve the information from the memory of the tag and transmit the same back to the mobile device 300. The controller 103 is arranged to energize the first antenna 107 to transmit information.

In an example operation, the mobile device 300 is brought into proximity with the electronics module 100. In response to this, the electronics module 100 is configured to energize the first antenna 107 to transmit information to the mobile device 300 over the first wireless communication protocol. Beneficially, this means that the act of the mobile device 300 approaching the electronics module 100 energizes the first antenna 107 to transmit the information to the mobile device 300.

The information may comprise a unique identifier for the electronics module 100. The unique identifier for the electronics module 100 may be an address for the electronics module 100 such as a MAC address or Bluetooth® address.

The information may comprise authentication information used to facilitate the pairing between the electronics module 100 and the mobile device 300 over the second wireless communication protocol. This means that the transmitted information is used as part of an out of band (00B) pairing process.

The information may comprise application information which may be used by the mobile device 300 to start an application on the mobile device 300 or configure an application running on the mobile device 300. The application may be started on the mobile device 300 automatically (e.g., without wearer 600 input). Alternatively, the application information may cause the mobile device 300 to prompt the wearer 600 to start the application on the mobile device. The information may comprise a uniform resource identifier such as a uniform resource location to be accessed by the mobile device, or text to be displayed on the mobile device for example. It will be appreciated that the same electronics module 100 can transmit any of the above example information either alone or in combination. The electronics module 100 may transmit different types of information depending on the current operational state of the electronics module 100 and based on information it receives from other devices such as the mobile device 300.

The second antenna 109 is arranged to communicatively couple with the mobile device 300 over a second wireless communication protocol. The second wireless communication protocol may be a Bluetooth® protocol, Bluetooth® 5 or a Bluetooth® Low Energy protocol but is not limited to any particular communication protocol. In the present embodiment, the second antenna 109 is integrated into controller 103. The second antenna 109 enables communication between the mobile device 300 and the controller 100 for configuration and set up of the controller 103 and the peripheral devices as may be required. Configuration of the controller 103 and peripheral devices utilises the Bluetooth® protocol.

The wireless communicator 159 may be an alternative, or in addition to, the first and second antennas 107, 109.

Other wireless communication protocols can also be used, such as used for communication over: a wireless wide area network (WWAN), a wireless metro area network (WMAN), a wireless local area network (WLAN), a wireless personal area network (WPAN), Bluetooth® Low Energy, Bluetooth® Mesh, Thread, Zigbee, IEEE 802.15.4, Ant, a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 includes configured a clock unit in the form of a real time clock (RTC) 153 coupled to the controller 103 and, for example, to be used for data logging, clock building, time stamping, timers, and alarms. As an example, the RTC 153 is driven by a low frequency clock source or crystal operated at 32.768 Hz.

The electronics module 100 also includes a location device 161 such as a GNSS (Global Navigation Satellite System) device which is arranged to provide location and position data for applications as required. In particular, the location device 161 provides geographical location data at least to a nation state level. Any device suitable for providing location, navigation or for tracking the position could be utilised. The GNSS device may include device may include Global Positioning System (GPS), BeiDou Navigation Satellite System (BDS) and the Galileo system devices.

The power source 105 in this example is a lithium polymer battery 105. The battery 105 is rechargeable and charged via a USB C input 131 of the electronics module 100. Of course, the present disclosure is not limited to recharging via USB and instead other forms of charging such as inductive of far field wireless charging are within the scope of the present disclosure. Additional battery management functionality is provided in terms of a charge controller 133, battery monitor 135 and regulator 137. These components may be provided through use of a dedicated power management integrated circuit (PMIC).

The USB C input 131 is also coupled to the controller 131 to enable direct communication with the controller 103 with an external device if required.

The controller 103 is communicatively connected to a battery monitor 135 so that that the controller 103 may obtain information about the state of charge of the battery 105.

The controller 103 has an internal memory 167 and is also communicatively connected to an external memory 143 which in this example is a NAND Flash memory. The memory 143 is used to for the storage of data when no wireless connection is available between the electronics module 100 and a mobile device 300. The memory 143 may have a storage capacity of at least 5 1 GB and preferably at least 2 GB.

The electronics module 100 also comprises a temperature sensor 145 and a light emitting diode 147 for conveying status information. The electronic module 100 also comprises conventional electronics components including a power-on-reset generator 149, a development connector 151, the real time clock 153 and a PROG header 155.

Additionally, the electronics module 100 may comprise a haptic feedback unit 157 for providing a haptic (vibrational) feedback to the wearer 600.

The wireless communicator 159 may provide wireless communication capabilities for the garment 200 and enables the garment to communicate via one or more wireless communication protocols to a remote server 500. Wireless communications may include: a wireless wide area network (WWAN), a wireless metro area network (WMAN), a wireless local area network (WLAN), a wireless personal area network (WPAN), Bluetooth® Low Energy, Bluetooth® Mesh, Bluetooth® 5, Thread, Zigbee, IEEE 802.15.4, Ant, a near field communication (NFC), a Global Navigation Satellite System (GNSS), a cellular communication network, or any other electromagnetic RF communication protocol. The cellular communication network may be a fourth generation (4G) LTE, LTE Advanced (LTE-A), LTE Cat-M1, LTE Cat-M2, NB-IoT, fifth generation (5G), sixth generation (6G), and/or any other present or future developed cellular wireless network.

The electronics module 100 may additionally comprise a Universal Integrated Circuit Card (UICC) that enables the garment to access services provided by a mobile network operator (MNO) or virtual mobile network operator (VMNO). The UICC may include at least a read-only memory (ROM) configured to store an MNO or VMNO profile that the garment can utilize to register and interact with an MNO or VMNO. The UICC may be in the form of a Subscriber Identity Module (SIM) card. The electronics module 100 may have a receiving section arranged to receive the SIM card. In other examples, the UICC is embedded directly into a controller of the electronics module 100. That is, the UICC may be an electronic/embedded UICC (eUICC). A eUICC is beneficial as it removes the need to store a number of MNO profiles, i.e. electronic Subscriber Identity Modules (eSIMs). Moreover, eSIMs can be remotely provisioned to garments. The electronics module 100 may comprise a secure element that represents an 35 embedded Universal Integrated Circuit Card (eUICC). In the present disclosure, the electronics module may also be referred to as an electronics device or unit. These terms may be used interchangeably.

The controller 103 is connected to the interface 101 via an analog-to-digital converter (ADC) front end 139 and an electrostatic discharge (ESD) protection circuit 141.

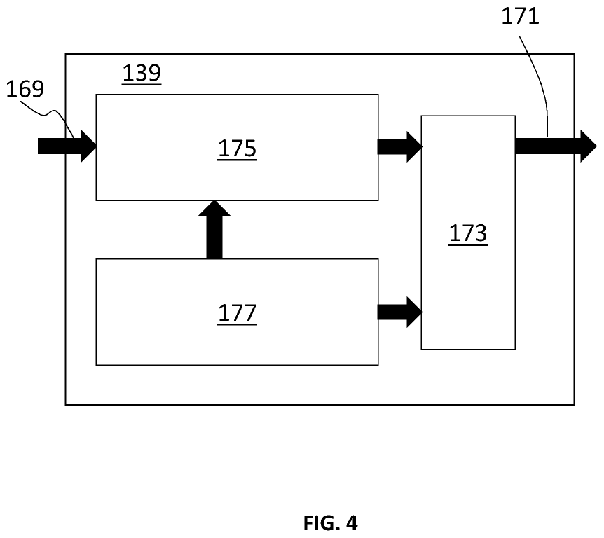

FIG. 4 is a schematic illustration of the component circuitry for the ADC front end 139.

In the example described herein, the ADC front end 139 is an integrated circuit (IC) chip which converts the raw analogue biosignal received from the sensors 209, 211 into a digital signal for further processing by the controller 103. ADC IC chips are known, and any suitable one can be utilised to provide this functionality. ADC IC chips for ECG applications include, for example, the MAX30003 chip produced by Maxim Integrated Products Inc.

The ADC front end 139 includes an input 169 and an output 171.

Raw biosignals from the electrodes 209, 211 are input to the ADC front end 139, where received signals are processed in an ECG channel 177 and subject to appropriate filtering through high pass and low pass filters for static discharge and interference reduction as well as for reducing bandwidth prior to conversion to digital signals. The reduction in bandwidth is important to remove or reduce motion artefacts that give rise to noise in the signal due to movement of the sensors 209, 211.

The output digital signals may be decimated to reduce the sampling rate prior to being passed to a serial programmable interface (SPI) 173 of the ADC front end 139.

ADC front end IC chips suitable for ECG applications may be configured to determine information from the input biosignals such as heart rate and the QRS complex and including the R-R interval. Support circuitry 177 provides base voltages for the ECG channel 175.

Signals are output to the controller 103 via the SPI 173.

The controller 103 can also be configured to apply digital signal processing (DSP) to the digital signal from the ADC front end 139.

The DSP may include noise filtering additional to that carried out in the ADC front end 139.

In the present embodiment, the controller 103 is configured to apply EMI filtering using a notch (or bandpass) filter implemented as a second-order 5-tap impulse response filters at 50 Hz and 60 Hz to filter EMI noise from the input ECG digital signal from the ADC front end. Alternatively filters with different orders and taps can be used. The filters are implemented using software to apply the relevant filtering function with the appropriate coefficients. Typically, the coefficients are based on the frequency to be filtered e.g., 50 Hz or 60 Hz and the ECG sampling rate e.g. 128, 256 or 512 samples per second.

Generally, the EMI noise will be dependent upon the utility frequency of the location of the electronics module 100 and the garment 200. In the UK, Europe and Australia, for example, mains supply is at 50 Hz, whereas North America mains supply is at 60 Hz.

The controller 103 is configured to apply the relevant notch filter depending upon the location. The internal memory 167 of the controller 103 stores the relevant filter coefficients for each of the different mains supply frequencies and applies the filter using the filter coefficients determined from the internal memory 167.

The mobile device 300 is configured to identify the location of the mobile device 300, for example using the mobile device's location capability, and to determine the relevant utility frequency in that location and to transmit a signal identifying the relevant utility frequency to the controller 103 via the second antenna 109, for example using Bluetooth. The controller 103 then determines the required filter coefficients and applies them to derive the appropriate filter for the utility frequency in the location.

Alternatively, the mobile device 300 may be configured to access details of the appropriate filter coefficients from a remote server (not shown) and then configured to transmit the filter coefficients directly to the controller 103.

In another alterative, the controller 103 includes a look-up table provided in the internal memory 167 which stores details of mains supply frequencies for relevant locations. The mobile device 300 is configured to identify the location of the mobile device 300, for example using the mobile device's location capability, and to transmit the location details to the electronics device 100. The controller 103 is then arranged to determine the relevant utility frequency from the look up table. The controller 103 is then operable to apply the appropriate filter with the required filter coefficients.

The geographical location need only be determined to a nation state level given that mains frequencies are used at this level.

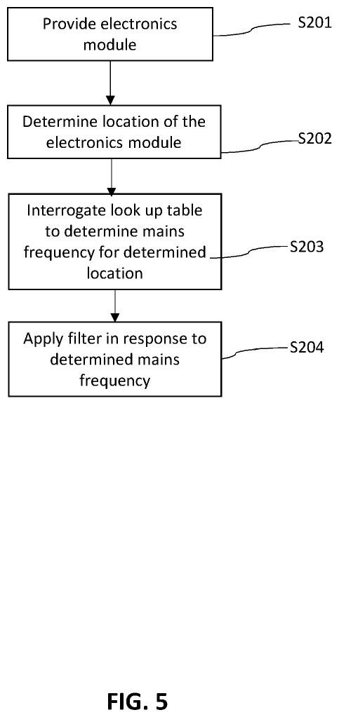

Referring to FIG. 5, there is shown a process flow diagram for an example method according to aspects of the present disclosure.

Step S201 of the method comprises providing an electronics module 100 such as the electronics module described above. In step S202, at start-up of the electronics module 100, or whenever the electronics module 100 is paired with the mobile device 300, the controller 103 interrogates the GPS device 161 to determine the location of the electronics module 100.

At step S203, the controller 103 interrogates a look-up table provided in the memory 167 to determine the utility frequency for the determined location. The look up table provides details of relevant mains frequencies for locations.

At step S204, the controller 103 is configured to apply the relevant notch filter for the determined location.

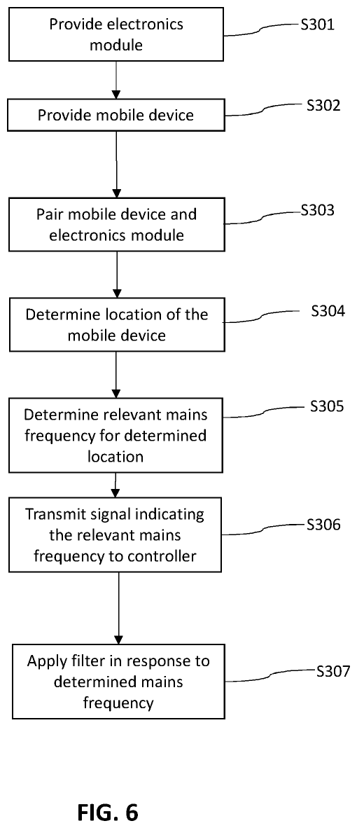

Referring to FIG. 6, there is shown a process flow diagram for another example method according to aspects of the present disclosure.

In this example, at step S301 an electronics module 100 such as the electronics module described above is provided. At step S302 a mobile device 300 such as the mobile described above is provided.

At step S303 the mobile device 300 and the electronics module 100 are paired, for example, using wireless transmission of information between the mobile device 300 and the electronics module 100 as describe above. Pairing, for example using Bluetooth® protocol is known to person skilled in the art.

At step S304, the mobile device 300 is configured to determine its location based on location tracking functionality provided on the mobile device 300. Such location tracking functionality is known to persons skilled in the art and uses, for example, a GNSS device on the mobile device 300.

At step S305, the mobile device 300 determines the utility frequency for the determined location.

At step S306, the mobile device 300 is configured to transmit a signal indicating the relevant utility frequency for the location, for example over the wireless communication using Bluetooth® protocol at the request of the controller 100. As an example, the mobile device 300 could be configured to send a binary “0” or “1” depending upon whether the relevant utility frequency is 50 Hz or 60 Hz. In an alternative and as described above, the mobile device could be configured to send the appropriate filter coefficients directly to the controller 103 at this step.

utility frequency. This assumes that the electronics module 100 and the mobile device 300 are co-located such that the location of the mobile device 300 and the location of the electronics module 100 are the same.

At step S307, the controller 103 is configured to apply the relevant notch filter for the determined location.

In a further embodiment, the controller 103 can be configured to be able to apply a filter at 400 Hz for when the electronic device 100 is being used within an airplane which has a utility frequency of 400 Hz. This could be done, for example, if the controller 103 receives a “flight mode” notification from the mobile device 300 indicating that the wearer 600 is on an airplane.

In yet another embodiment, the controller 103 could be configured to apply other filters, and in particular other noise filters, if such a selection might be dependent upon, or influenced by location. For example, the location device 161 may provide location data to a very specific location such as a care home or a hospital, a training facility, or home. As discussed above, clinical settings may require other noise artefacts to be removed or mitigated against. In this example, therefore if the location is deemed to be that of a clinical setting, other relevant filters such as those for diagnostic or monitoring purposes, can be selected and applied.

In yet a further embodiment, the location device 161 will provide location data indicating that the user has left a building and, as such, electromagnetic interference is less likely to be an issue. The controller 103 may, in these circumstances, be configured to not apply the filter.

In some embodiments, the described elements may be configured to reside on a tangible, persistent, addressable storage medium and may be configured to execute on one or more processors. These functional elements may in some embodiments include, by way of example, components, such as software components, object-oriented software components, class components and task components, processes, functions, attributes, procedures, subroutines, segments of program code, drivers, firmware, microcode, circuitry, data, databases, data structures, tables, arrays, and variables.

Although the example embodiments have been described with reference to the components, modules and units discussed herein, such functional elements may be combined into fewer elements or separated into additional elements. Various combinations of optional features have been described herein, and it will be appreciated that described features may be combined in any suitable combination. In particular, the features of any one example embodiment may be combined with features of any other embodiment, as appropriate, except where such combinations are mutually exclusive. Throughout this specification, the term “comprising” or “comprises” means including the component(s) specified but not to the exclusion of the presence of others.

All of the features disclosed in this specification (including any accompanying claims, abstract and drawings), and/or all of the steps of any method or process so disclosed, may be combined in any combination, except combinations where at least some of such features and/or steps are mutually exclusive.

Each feature disclosed in this specification (including any accompanying claims, abstract and drawings) may be replaced by alternative features serving the same, equivalent or similar purpose, unless expressly stated otherwise. Thus, unless expressly stated otherwise, each feature disclosed is one example only of a generic series of equivalent or similar features.

The invention is not restricted to the details of the foregoing embodiment(s). The invention extends to any novel one, or any novel combination, of the features disclosed in this specification (including any accompanying claims, abstract and drawings), or to any novel one, or any novel combination, of the steps of any method or process so disclosed.

1-23. (canceled)

24. A method performed by a controller for an electronics module for a wearable article, the electronics module further including an interface, coupled to the controller, and arranged to receive signals from a sensor unit, wherein the method comprises determining the location of the electronics module, and selecting a filter for the controller to apply to the received signal based on the determined location.

25. The method according to claim 24, wherein the method comprises determining a likely frequency of electromagnetic interference present in a received signal from the determined location and selecting the filter for the controller based on the determined location and the determined likely frequency.

26. The method according to claim 24, wherein the method comprises determining a likely use of the electronic module from the determined location and selecting the filter for the controller based on the determined location and the determined likely frequency.

27. The method according to claim 26, wherein the likely use is determined to be in a clinical setting.

28. The method according to claim 26, wherein the likely use is determined to be in an outside setting and the method comprises selecting a filter in response to determining a location of an outside setting.

29. The method according to claim 24, wherein the method comprises accessing a look up table stored in memory on the electronics module to determine the likely frequency for the determined location.

30. The method according to claim 25, wherein the likely frequency is a utility frequency.

31. The method according to claim 24, wherein the location is derived from a location device.

32. The method according to claim 31, wherein the location is derived from a location device provided on the electronics module.

33. The method according to claim 31, wherein the location is derived from a location device provided on a mobile device in communication with the electronics module, the method including the step of receiving the location data from the mobile device prior to determining the likely frequency of electromagnetic interference present in a received signal from the determined location.

34. The method according to claim 24, wherein the location is derived from data obtained from a Global Navigation Satellite System.

35. An electronics module for a wearable article, the electronics module comprising a controller and an interface, coupled to the controller, and arranged to receive signals from a sensor unit, wherein the controller is configured to determine the location of the electronics module, and to select a filter for the controller to apply to the received signal based on the determined location.

36. The electronics module according to claim 35, wherein the controller is further configured to determine a likely frequency of electromagnetic interference present in a received signal from the determined location, and to select the filter for the controller based on the determined location and the determined likely frequency.

37. The electronics module according to claim 35, wherein the controller is further configured to determine a likely use of the electronic module from the determined location and selecting the filter for the controller based on the determined location and the determined likely frequency.

38. The electronics module according to claim 37, wherein the likely use is determined to be in a clinical setting.

39. The electronics module according to claim 37, wherein the likely use is determined to be in an outside setting and the method comprises deselecting a filter in response to determining a location of an outside setting.

40. The electronics module according to claim 35, wherein the electronics module further comprises a memory including a look up table and the controller is configured to access the look up table to determine the likely frequency for the determined location.

41. The electronics module according to claim 36, wherein the likely frequency is a utility frequency.

42. The electronics module according to claim 35, and further comprising a location device coupled to the controller and arranged to provide location data to the controller, whereby the controller is configured to determine the location from the location data.

43. The electronics module according to claim 35, and further comprising a communicator coupled to the controller and arranged for communication with a mobile device, whereby the determined location is derived from a location device provided on the mobile device.

44. The electronics module according to claim 43, wherein the location device is a Global Navigation Satellite System device.

45. A wearable article including an electronics module according to claim 35.

46. A controller for an electronics module according to claim 45.
